(19) United States
(12) Patent Application Publication
(10) Pub. No.: US 2005/0063940 A1
(43) Pub. Date: Mar. 24, 2005

(54) Bioadhesive agent

(76) Inventor: Sveinbjorn Gizurarson, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/505,584

(22) PCT Filed: Feb. 24, 2003
(86) PCT No.: PCT/IS03/00009

(30) Foreign Application Priority Data
Feb. 25, 2002 (IS) 6283
Jul. 3, 2002 (IS) 6453

(51) Int. Cl. A61K 31/765

(52) U.S. Cl. 424/78.38

(57) ABSTRACT

One or more mono- or diglycerides according to formula I: wherein R1, R2, and R3 are selected from the group consisting of from C6 to C26 fatty acids, PEG polymers and hydrogen, provided that at least one of R1, R2 and R3 is a C6-C26 fatty acid residue and at least one of R1, R2 and R3 is a PEG polymer residue for use as a bioadhesive agent for medical purposes. The agent may be used in the delivery of e.g. protein, peptides or antigens over mucosal membranes.

### FIELD OF THE INVENTION

The present Invention relates to the use of mono- and diglycerides according to formula I as bioadhesive agents. The agents can be used to prolong the residence time of an active substance at the administration site and, thus, in certain cases reduce the dose of the active substance. The bioadhesive agents are extremely water-soluble which enables the preparation of aqueous pharmaceutical compositions having a single phase.

### BACKGROUND OF THE INVENTION

Parenteral administration (Intramuscular and subcutaneous) of therapeutically, prophylactically and/or diagnostically active agents such as drug, protein, peptide, ion, gene, plasmids, antisense molecule, oligonucleotides, diagnostics, antibodies (therapeutic and/or prophylactic), antigens such as vaccines or allergens Is normally regarded as the most effective route of administration. However, administration by injection has a number of disadvantages. Injection of an active agent requires the use of sterile syringes and administration by trained personnel, and may cause pain and Irritation, particularly in the case of repeated injections. This route of administration also poses a risk of Infection. More significantly, intramuscular Injections are often poorly tolerated by the individual, and may possibly cause an Induration (hardening of tissue), haemorrhage (bleeding) and/or necrosis (local death of tissue) at the injection site.

The mucosal membrane is connected to an extensive network of blood capillaries, for example under the nasal mucosa, which makes the membrane highly suitable for drug delivery (delivery of active agents), particularly suited to provide rapid absorption of active agents, providing a quick therapeutic, prophylactic and/or diagnostic response. One example of such a mucosal membrane is the nasal epithelial membrane, which has a single layer of epithelial cells (pseudo-stratified epithelium); the mucosal membrane Is therefore very suitable for drug delivery.

A variety of vehicle systems for intranasal drug delivery have been developed. One of the problems encountered in using such vehicle systems, is the short time at the site of absorption. Due to the rapid clearance from most mucosal surfaces, the active agents, such as drugs or vaccines, may be cleared from the absorption site before they are absorbed into the systemic circulation, into the lymphatic system or into the brain. Known formulations containing e.g. polyethylene glycols, propylene glycols etc. are normally viscous and provide a mechanical barrier for the natural clearance mechanism.

Its contemplated that a vehicle that is bioadhesive as well as water-soluble would be an advantage for mucosal administration. However, to the best of the present inventors knowledge no such vehicle has yet been found. Thus, there is a need for developing a bioadhesive agent, which may be used especially in hydrophilic compositions. It is also an advantage if it can be used in lipophilic compositions as well. The bioadhesive agent should be suitable for mucosal delivery of active substances such as drug substances and/or vaccines.

A variety of vehicle systems for intranasal drug delivery have been developed. One of the problems encountered in using such vehicle systems, is the local irritation and malabsorption. A frequent problem is that the substance may be cleared from the absorption site before it may be absorbed into the systemic circulation, into the lymphatic system or into the brain. Many excipients such as polyethylene glycol and glycofuroium (Bechgaard, Gizurarson & Hjortkjaer, DK-1170/90 and Bechgaard, Gizurarson & Hjortkjaer, U.S. Pat. No. 5,397,771) are highly viscous and therefore not suitable for the purpose. Thus, there is a need for an effective formulation for intranasal or mucosal drug delivery that may be used in low concentration without being affected by the viscosity.

WO 99/02186 describes antigen delivery systems comprising monoglyceride or diglyceride derivatives as adjuvants. The monoglyceride or diglyceride derivatives mentioned therein may contain a PEG polymer. However, there is no mention or indication that such derivatives are capable of adhering to a mucosal surface.

U.S. Pat. No. 6,326,401 relates to formulation for the oromucosal in particular the pernasal route. It describes pharmaceutical compositions comprising caprylcaproyl-macrogol glycerides for delivery of non-polypeptidic active substances. There is no mention or indication that the glycerides have bioadhesive properties.

### SUMMARY OF THE INVENTION

The present invention is based on the observation that mono- and diglycerides according to formula I are bioadhesive. Furthermore, the mono- and diglycerides are water-soluble which means that the bioadhesive properties are present in a monophasic system such as an aqueous based system. This observation of a bioadhesive effect is extremely valuable in respect of design of pharmaceutical composition for administration to mucosal surfaces. Firstly, it is possible to ensure that the composition after administration will remain on the administration site for a prolonged period of time (i.e. a rapid clearance effect can be avoided) and, secondly, due to the water-solubility of the glycerides it is possible to formulate the compositions as a single phase composition, i.e. it is not necessary to add any surfactants (including emulsifiers) etc in order to stabilize a two or more phase composition and thereby it is possible to avoid any irritating effect arising from such surfactants.

Thus the present invention relates to the use of a composition comprising
one or more mono- or diglyceride having the formula (I): 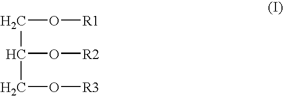
   wherein R1, R2, and R3 are selected from the group consisting of from C6 to C26 fatty acids, PEG polymers and hydrogen,
provided that at least one of R1, R2 and R3 is a C6-C26 fatty acid residue and at least one of R1, R2 and R3 is a PEG polymer residue
as a bioadhesive agent.

The bioadhesive agent is suitable for administration of active substances, such as drug, protein, peptide, ion, gene, plasmids, antisense molecule, oligonucleotides, diagnostics, antibodies (therapeutic and/or prophylactic), antigens such as vaccines or allergens to mucosal membranes, particularly the nasal, olfactory, buccal, rectal, otal, ocular, vaginal membrane or to the skin, nail, hair or to the gills of fish or to plants. Use according to the invention provides a bioadhesive effect providing longer duration of the active substance at the mucosal membrane. Use of such bioadhesive compositions provides the ability to achieve a significantly prolonged duration at the site of absorption of biologically active agents such as drugs, protein, peptide, ion, gene, plasmids, antisense molecule, oligonucleotides, diagnostics, antibodies (therapeutic and/or prophylactic), antigens such as vaccines or allergens without causing unacceptable irritation of the epithelial membrane.

In another aspect, the invention relates to a method for the preparation of a bioadhesive pharmaceutical composition comprising mixing a bioadhesive agent according to the invention with an active substance optionally together with one or more pharmaceutically acceptable excipients.

In a further aspect, the invention relates to a method for the preparation of a bioadhesive composition for use in plants comprising mixing a bioadhesive agent according to the invention with an active substance selected from the group consisting of herbicides, insecticides, fungicides, plant growth regulators, fertilizers, antigens and vaccines.

In other aspects, the invention relates to a method to prolonging the duration of an active substance at outer surfaces of a mammal by administration of the active substance with a bioadhesive agent according to the invention, and to a kit comprising a first and a second component, the first component comprising a bioadhesive agent according to the invention and the second component comprising an active substance.

### DETAILED DESCRIPTION OF THE INVENTION

For nasal, olfactory, buccal, ocular, otal, vaginal, rectal, dermal, nail or hair administration, a therapeutically, prophylactically and/or diagnostically active agent must be applied to the surface in such a manner that it is delivered to the mucosal surface. The bioadhesive agent will protect the biologically active substance to be washed away e.g. by the nasal secretions, gravity and/or ciliary beat clearance mechanism. The bioadhesive agent is able to provide prolonged duration of active substances such as proteins, peptides, small drugs as well as larger molecules having hydrophilic and/or hydrophobic characteristics. That is, in certain cases, the active agent may remain on the mucosa for at least 2-16 hours such as 2-6 hours. This time period is sufficient for active substance with poor bioavailability.

Surprisingly, it has been found by the present inventor that administration of a therapeutically, prophylactically and/or diagnostically active substance together with a bioadhesive agent according to the invention will result in a response that is prolonged or increased significantly due to the bioadhesive effect provided by the bioadhesive agent it is contemplated that a dose reduction may be possible when a bioadhesive composition is applied.

Similarly a vaccine may be kept at the site of absorption for significantly longer time, providing the possibility for low dose administration as well as enough time for macrophages, dendritic cells or Langerhans calls to recognize the antigen.

As mentioned above, the present invention relates to the use of a composition comprising
one or more mono- or diglyceride having the formula (I): 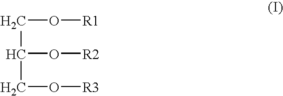
   wherein R1, R2, and R3 are selected from the group consisting of from C6 to C26 fatty acids, PEG polymers and hydrogen,
provided that at least one of R1, R2 and R3 Is a C6C26 fatty acid residue and at least one of R1, R2 and R3 is a PEG polymer residue
as a bioadhesive agent.

The PEG polymer on the glycerides imparts the desired water-solubility to the bioadhesive agent. Thus, it the water-solubility of the bioadhesive agent is relatively high, i.e. at least 40% w/w such as at least 50% w/w or at least 60% w/w. As mentioned above the high water-solubility makes it possible to design pharmaceutical compositions in liquid form that are in the form of single phase aqueous systems and thereby it is possible to avoid side effects related to the presence of surfactants or other additives that stabilize e.g. emulsions or suspensions.

Any saturated or unsaturated C6-26 fatty acid can be used including, but not limited to, fatty acid residues derived from caproic acid, capric acid, caprylic acid, arachidonic acid, propionic acid, lauric acid, myristic acid, palmitic acid, oleic acid, linoleic acid and linolenic acid. Examples of suitable fatty acids are saturated or unsaturated C6-20, C6-C18, C6-14, C6-12, C8-12 or C8-10 fatty acids. In one embodiment, the fatty acids are C6, C8 or C10 fatty acids.

The fatty acid residues may be a single residue or a mixture of two or more residues. They can be derived from natural or synthetic sources, such as fats and oils. Commercially available glycerides can be used or they can be synthesized enzymatically by means of lipases, including both specific and non-specific lipases, which place the fatty acids on specific positions (R1, R2 and/or R3) such as R1; R2; R1, R2; R1, R3; and specific racemic structures as well, etc. For example, glycerol (0.92 g) adsorbed onto 1 g silica gel is suspended with 20 ml tBuOMe. Vinyl caprylate (3.4 g) and lipase (50 mg) from Rhizopus delemar were added to the suspension. The mixture was stirred at room temperature for 96 hours and the reaction progress was monitored by means of TLC. After removal of the solid components by filtration and evaporation of the solvent, a crude reaction mixture was obtained which contained about 91% of 1,3 dicaprylin glyceride

A suitable PEG polymer is one that is biocompatible with the tissue to which it is administered, particularly the mucus membranes. Suitable PEG polymers having these properties include but are not limited to, polyethylene glycol, polyoxyethylene glycol, polyethylene glycol derivatives (including, but not limited to, amino-PEG, nucleophilic-PEG, PEG-thiol, PEG-succinate, PEG-succinimide, PEG-tresylate, carboxymethylated-PEG, PEG-propionic acid, PEG-silanes, PEG-phospholipids, biotin-PEG and PEG-orthopyridyl-disulfide) and polyoxyethylene glycol derivatives.

Preferred are polyethylene glycol polymers having from about 2 to about 30 polyethyleneglycol units (PEG2-30) or PEG polymers that have from 2 to about 15 residues such as, e.g., from 2 to about 10 residues, from 2 to about 8 residues or from about 3 to about 6 residues.

One or two PEG moieties can be incorporated into the glyceride formula. The water-soluble moiety can reside at any one of the R1, R2, and/or R3 positions of the glyceride.

Thus, the composition for use according to the invention may comprise a mixture of at least a first and a second glyceride, the first glyceride having one PEG polymer in position R1, R2 or R3, and the second glyceride having two PEG polymers in position R1, R2 and/or R3.

The polymers can be attached to the glyceride via covalent bonds formed chemically or enzymatically. The polymers may be acylated to the glyceride or linked using ester bonds such as, for example, by esterase-mediated synthesis. For example, solketal can be mixed with polymerchloride in triethanolamine and trichloromethane, whereafter the free glycerol bonds are deprotected by heating in dilute aqueous acetic acid. With excess of caproyl chloride in the presence of triethylamine and 4-dimethylaminopyridine as catalyst, the fatty acids are linked to the free glycerol bonds. The results of this synthesis will be a caproyl/polymer glyceride.

In one embodiment, the composition for use according to the invention comprises a mixture of mono- and diglycerides having formula I, such as a mixture of one or more monoglycerides and/or a mixture of one or more diglycerides. The vv-% ratio of monoglycerides to diglycerides being from about 0.1:99.9 to about 99.9:0.1 such as, e.g., from about 1:99 to about 99:1, from about 2.5:97.5 to about 97.5:2.5, and preferably from about 5:95 to about 95:5.

In a second embodiment, the mono- or diglyceride has a structure selected from the group consisting of:

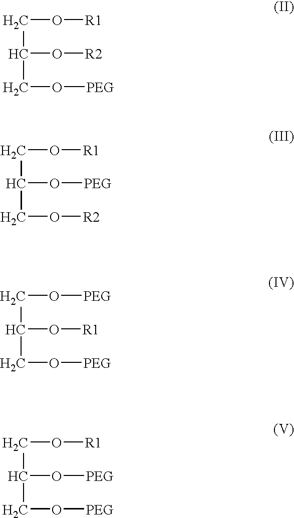

The PEG polymers in formulas II-V have from about 2 to about 30 polyethylene glycol units (PEG2-30). In a specific embodiment, the PEG in formula II-V is selected from the group consisting of PEG2, PEG3, PEG4, PEG5, PEG6, PEG7, PEG8, PEG9, PEG10, and in yet another specific embodiment, the PEG in formula II-V is PEG3 or PEG6.

In another embodiment, the monoglyceride has the following structure 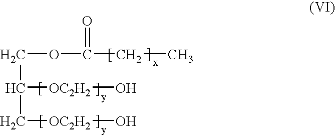
and in yet another embodiment, the diglyceride has the following structure

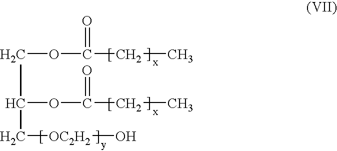

In a specific embodiment, the composition contains a mixture of the two following structures

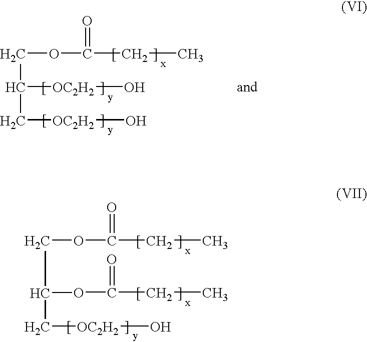

In the formulas VI and VII, x is from about 4 to about 20, such as e.g. from about 4 to about 12, or from about 6 to about 8, and y is from 2 to about 30, such as e.g. from 2 to about 10, or from 3 to about 6. In a specific embodiment of the invention, x is 6 and/or 8 and y is 3 and/or 6.

The total concentration of glycerides of formula I in the composition is at-least about 90% w/w such as, e.g., at least about 92.5% w/w, at least about 95% w/w, at least about 97.5% w/w, at least about 98% wtw or at least about 99% w/w and is normally from about 0.05-99.95 of glyceride fatty acid diester of formula II and III such as, e.g. from about 5 to about 95% glyceride fatty acid die sters of formula II and III and from about 0.05 to about 99.95% glyceride fatty acid mono esters of formula IV and V such as, e.g. from about 5 to about 95% glyceride fatty acid monoesters of formula IV and V.

In one embodiment, the glyceride has a chiral carbon, and in a specific embodiment the chiral carbon is S— or R-form.

The bioadhesive agent according to the present invention can be used to prolonging the duration of the active substances at the surface of a vertebrate such as a mammal or a human.

Thus, the invention furthermore relates to a method to prolonging the duration of an active substance at the outer surface, such as a mucosal surface of a mammal by administration of the active substance with a bioadhesive agent according to the invention. Furthermore, an active substance can be combined with one or more of the bioadhesive agents according to the invention.

In addition to combining an active agent with a bioadhesive agent according to the invention, the active agent may further be combined with one or more of the absorption promoting agents.

The active substance can be selected from the group including, but not limited to materials having antiviral, antiprion, antibacterial, antineoplastic, antiparasitic, anti-inflammatory and/or antifungal activity. They may act as neurotransmitter, neuromodulators, nootropic, hormone, hormone releasing factor, hormone receptor agonist or antagonist. The agent may also be an activator or inhibitor of a specific enzyme, an antioxidant, a free radical scavenger, a metal chelating agent, or an agent that alters the activity of ion channels of brain cell membranes, for example nimodipine. The active substance may further be any substance which is capable of acting as a stimulant, sedative, hypnotic, analgesic, anticonvulsant, antiemetic, anxiolytic, antidepressant, tranquilliser, cognition enhancer, agents preventing or healing amnesia, metabolic stimulator or inhibitor, appetite stimulator or inhibitor and/or narcotic antagonist or agonist. The substance may furthermore be any bioactive material found to be deficient in conjunction with the disorder being treated or prevented, for example, nutrients such as glucose, ketone bodies, and the like, or metabolic precursors such as lecithin, choline or acetyl coenzyme A for producing neurotransmitters for the treatment of Alzheimer's disease or insulin for the treatment of obesity and diabetes. Proteins and peptides are especially suitable for this system. The substance may also be an antibody for the treatment of viral, bacterial, prion, parasitic infections or tumours and/or cancer or for diagnosis of diseases or disorders where polyclonal or monoclonal antibodies and/or with biochemical markers characteristic of the diseases or disorder are used. Such diagnostic antibodies may be labelled with any labelling agent who may be suitable according to the invention. Gene manipulated microorganisms may also be used for the treatment of tumours and/or cancer. The active substance may also comprise of substances selected from the group consisting of adrenal hormones, corticosteroids and derivatives, anabolic steroids such as testosterone and derivatives, amino acids, anorectics, antibiotics, anti-allergic agents, antibodies, anti-cholinergic agents, anti-depressants, anti-emetica, anti-epileptica and anti-spasmolytica, anti-histaminic agents, anti-hypertensive agents, anti-inflammatory agents (enzymatic or non-steroidal or steroidal), anti-neoplastic agents, antiseptics, anti-tumor, anti-tussive expectorant (asthmatic agents), anti-viral and anti-cancer agents, beta-adrenergic blocking agents, blood factors such as factor VII, factor VII etc, metabolism controlling agents, bone-metabolism controlling agents, bronchoister, cardiotonics, cardiovascular regulatory hormones, chemoterapeutic agents, CNS-stimulants, corticosteroids, diagnostic drugs, dopaminergic agents, enzymes, gastrointestingal hormones, hypothalamus hormones and derivateves, hypotensives, local anesthetics, migraine treatment substances, narcotics, antagonists and analgetics, pancreatic hormones and derivatives, parasympathomimetics, parasympatholytics, Parkinson disease substances, pituitary gland hormones and derivatives, prostaglandines, protease inhibitors, sedatives, sex-hormones, sympathomimetics, thyroid gland hormones and derivatives, tranquilliser, vasoconstrictors, vasodilators, and vitamins.

The bioadhesive effect of a bioadhesive agent according to the invention, with a particular active agent, can be assessed using methods known in the art, such as bioadhesiometer, radioactive tracers, fluorescence tracers. As used herein, “bioadhesive effect” is intended to mean the ability to increase the duration of an active substance at the site of absorption. Bioadhesive effects include, but are not limited to, the ability to prolong the duration of the active agent by decreasing the clearance of the active agent from the site of absorption, such as the nasal cavity.

Typically the administration of the bioadhesive agent of the invention will cause or result in a controlled release and prolonged pharmacological response to an active agent of interest. In this context, “prolonged” is intended to mean that the pharmacological effect of an active agent is quantitatively longer and/or therefore qualitatively better in the presence of the bioadhesive agent of the invention than in the absence of the bioadhesive agent. Comparisons of the effect in the presence and absence of the bioadhesive agent can be performed by routine methods, such as monitoring the clearance of radiolabelled tracers or fluorescence labelled tracers and an appropriate control. The prolonged duration can be a result of a biocompatibility with the mucosal environment and/or direct effect on the mucosal membrane of due to a more advantageous presentation of the active agent to the mucus membrane.

Typically the bioadhesive agent will be administered together with an active substance, either in the same admixture or composition, or at the same time but in a separate composition or formulation. The bioadhesive agent can also be administered up to 12 hour prior to or subsequent to the administration of the biologically active agent.

Thus, the present invention also relates to a kit comprising a first and a second component, the first component comprising a bioadhesive agent according to invention and the second component comprising an active substance. In one embodiment, the first and the second component are administered at substantially the same time. In another embodiment, the first and second component are administered sequentially, up to 12 h apart, preferably 4 hours and more preferably 2 h apart, the first component being administered firstly.

The invention further relates to a method for the preparation of a bioadhesive pharmaceutical composition comprising mixing a bioadhesive agent according to the invention with an active substance optionally together with one or more pharmaceutically acceptable excipients. In one embodiment, the active substance is a drug (i.e. testosterone), protein, peptide, ion, gene, plasmids, antisense molecule, oligonucleotides, diagnostics, antibodies (therapeutic and/or prophylactic), antigens such as vaccines, autoimmune diseases antigens, allergens, substance antigen such as environmental toxins, narcotics, drugs, proteins for the treatment of Alzheimer's etc. or a cancer antigen. Other types of active substances are described above.

The active substance such as drugs may be used in a particulate form or in a dissolved form. The formulation is especially suitable for dissolved drugs. However, particulate forms are also easy to prepare into the formulation.

The amount of active substance employed together with the bioadhesive agent will vary depending upon the identity of the active substance. Adjustment and manipulation of established dosage ranges used with desired pharmacological responses, which is within the ability of those skilled in the art.

The bioadhesive agent has a concentration of from about 0.005% to about 99% by weight of a pharmaceutical composition prepared according to the invention, such as e.g. from about 0.1% to about 99%, or from about 0.5% to about 20%, or from about 1% to about 15%. In a specific embodiment, the bioadhesive agent has a concentration of from about 0.005% to about 50% by weight of the pharmaceutical composition, such as e.g. from about 0.01% to about 20%, or from about 0.01% to about 10%, or from about 0.01% to about 5%, or from about 0.01% to about 2%, or from about 0.1% to about 1.5%, or from about 0.2% to about 1%.

The method of the present invention comprises administering to a mammal, particularly a human or other primate, a pharmacologically effective dose of an active substance composition comprising an active substance and a bioadhesive agent according to the invention. For example, doses of from about 0.1% to about 20%, and more particularly from about 0.5% to about 5% will typically be effective to provide a bioadhesive effects; however, variations in these dosage ranges will occur depending upon the particular bioadhesive agent and the biologically active agent. Moreover, the particular dosage will depend upon the age, weight and medical condition of the mammal to be administered, as well as on the method of administration.

Pharmaceutically acceptable exciplents may include surfactants e.g. emulsifying agents, absorption promoters, water absorbing polymers, substances which inhibit enzymatic degradation, alcohols, organic solvents, oils, pHcontrolilng agents, solubilizers, stabilizers, HLB-controlling agents, viscosity controlling agents, preservatives, osmotic pressure controlling agents, propellants, antioxidants, buffering agents, humectants, penetration enhancers, chelating agents, gelforming agents, ointment bases, perfumes, and skin protective agents, air displacement, water, and mixtures thereof.

Examples of emulsifying agents are naturally occurring gums, e.g. gum acacia or gum tragacanth, naturally occurring phosphatides, e.g. soybean lecithin, and sorbitan monooleate derivatives. Examples of antioxidants are burylated hydroxy anisole (BHA), ascorbic acid and derivatives thereof, tocopherol and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone, cholic acids, glycocholic acids, EDTA, glycofurols, fusidic acids, laureth-9 and derivatives thereof, tetrahydrofurfuryl alcohol, and Azone. Examples of chelating agents are sodium EDTA citric acid, and phosporic acid.

Examples of other excipients are edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil; and of polymers such as carmelose, sodium carmelose, hydroxypropylmethylcellulose, hydroxyethylcellylose, hydroxypropylcellulose, chitosane, pectin, xanthan gum, carragenan, locust bean gum, acacia gum, gelatin, and alginates. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

In one aspect, the invention relates to a method of eliciting a therapeutic, prophylactic and/or diagnostic effect in a mammal, comprising administering to the mammal an effective amount of a composition according to the invention.

The composition according to the invention can be optionally administered in a pharmaceutically or physiologically acceptable vehicle, such as physiological or phosphate buffered saline, water, dextrose, ethanol polyols (such as glycerol or propylene glycol), and combinations thereof. The formulation according to the invention can be a suspension, an emulsion or dispersion and can provide the bioadhesive agent in admixture with a dispersing or wetting agent, suspending agent, and/or one or more preservatives. Suitable dispersing or wetting agents are, for example, naturally occurring phosphatides, e.g., lecithin, or soybean lecithin; condensation products of ethylene oxide with e.g. a fatty acid, a long chain aliphatic alcohol, or a partial ester derived from fatty acids and a hexitol or a hexitol anhydride, for example polyoxyethylene stearate, polyoxyethylene sorbitol monooleate, polyoxyethylene sorbitan monooleate etc. Suitable suspending agents are, e.g., naturally occurring gums such as, e.g., gum acacia, xanthan gum, or gum tragacanth; celluloses such as, e.g., sodium carboxymethylcellulose, microcrystalline cellulose (e.g. Avicel RC 591), methylcellulose; alginates such as, e.g., sodium alginate, etc. Suitable examples of preservatives for use in formulations according to the invention are parabens, such as methyl or propyl p-hydroxybenzoate, and benzalkonium chloride.

In another aspect, invention relates to a method of delivering an active agent to a mammal, comprising administering to a surface of the skin, nail, hair or to a mucosal surface of the mammal an effective amount of a composition according to the invention. In a specific embodiment, the invention relates to a method of delivering an active agent to a mucosal surface of a mammal, and in yet another embodiment, the mucosal surface is selected from the group of mucosa surfaces of the nose, lungs, mouth, eye, ear, gastrointestinal tract, genital tract, vagina, rectum

The invention also relates to a method, wherein the administration of the composition of the invention leads to absorption of the active substance into the systemic circulation, the lymphatic circulation or the brain, e.g. via the olfactory route.

For application to the rectal or vaginal mucosa, suitable formulations for use according to the invention include suppositories (emulsion or suspension type), enemas, and rectal gelatin capsules (solutions or suspensions). Appropriate pharmaceutically acceptable suppository bases include cocoa butter, esterified fatty acids, glycerinated gelatin, and various water-soluble or dispersible bases like polyethylene glycols and polyoxyethylene sorbitan fatty acid esters. Various additives, e.g., enhancers or surfactants, can also be incorporated.

For application to the nasal mucosa, nasal sprays and aerosols for inhalation are suitable compositions for use according to the invention. In a typical nasal formulation, the active substance is present in the form of a particulate formulation optionally dispersed in a suitable vehicle. The pharmaceutically acceptable vehicles and excipients and optional other pharmaceutically acceptable materials present in the composition such as diluents, enhancers, flavouring agents, preservatives and the like are all selected in accordance with conventional pharmaceutical practice in a manner understood by the persons skilled in the art.

Other surfaces which are suitable for the administration of formulations of the invention are the nose, lungs, mouth, eye, ear, gastrointestinal tract, genital tract, vagina, rectum, skin, nails or hair, or in fish to the gills. Such formulations may also be suitable for application to seeds or leaves of plants.

For application to the skin, nail or the hair, the formulations according to the invention may contain conventional non-toxic pharmaceutically acceptable carriers and excipients Including micro-spheres and liposomes. The formulations include creams, ointments, lotions liniments, gels, hydrogels, solutions, suspensions, sticks, sprays, pastes, plasters, and other kind of transdermal drug delivery systems. In one embodiment, the formulation can be administered as nasal spray, nasal drops, nasal powder, nasal foam or as nasal ointment to the mucosal surface or the adenoids of the nose or as oral spray, oral drops, oral powder, oral foam or as oral ointment especially directed to the buccal area or to the tonsils of the mouth. The formulation can also be in the form of eye drops.

Formulations of the invention may also be suitable for direct application or for introduction into relevant orifice(s) of the body, e.g. the rectal, urethral, vaginal or oral orifices. The formulation may simply be applied directly on the part to be immunized such as, e.g., the mucosa.

Many mucosal formulations need some specialized mixture of excipients. Therefore many formulations may comprise one or more surfactants and/or absorption promoters and/or water absorbing polymers and/or substances which inhibit enzymatic degradation and/or alcohols, organic solvents, oils, pH-controlling agents, solubilizers, stabilisers, HLB-controlling agents, viscosity controlling agents, preservatives, osmotic pressure controlling agents, propellants, air displacement, water and mixture thereof. The surfactants may be selected from nonoxynol, octoxynol, Tweens, spans sodium lauryl sulfate, sorbitan monopalmitate; absorbing promoters may be selected from polyoxyethylene alcohol ethers, bile salts and derivatives thereof, fusidic acid and derivatives thereof, oleic acid, lecitin, lysolechitins, Tweens 20-85, mono-/di- and triglycerides, chitosan, cyclodextrins, water absorbing polymers may be selected from glycofurols and derivatives thereof, polyethylene glycol 200-7500 and derivatives thereof, polyvinylpyrrolidone, polyacrylic acid, propylene glycol, gelatine, cellulose and derivatives thereof, substances which inhibit enzymatic degradation may be selected from aprotinin, DFP, carbopol; oils may be selected from vegetable oil, soybean oil, peanut oil, coconut oil, maize oil, olive oil, sunflower oil, Miglyols; pH-controlling agents may be selected from acetic acid, hydrochloric acid, nitric acid, potassium metaphosphate, potassium phosphate, sodium acetate, ammonia, sodium carbonate, sodium hydroxide, sodium borate, trolamine; solubilizers may be selected from alcohol, isoporpyl alcohol, water glycofurol, polyethyleneglycole 200-7500; stabilisers such as cyclodextrins; HLB controlling agents may be selected from Tween 20-85, Span 20- 80, Brij 3098, acacia; viscosity controlling agents may be selected from cellulose and derivatives, thereof, Tweens and derivatives thereof, polyethyleneglycole and derivatives thereof, cetyl alcohol, glycerine, propylene glycol, sorbitol, gelatin; preservatives may be selected from benzalkonium salt, benzyl alcohol, phenol, thimerosal, phenylmercuric nitrate, phenylethyl alcohol, chlorobutanol, cetypyridinium chloride; osmotic pressure controlling agents may be selected from dextrose, sodium chloride, mannitol; and propellants may be selected from dichlorodifluoromethane, dichlorotetrafluoroethane, trichloromonofluoromethane and other non-ozone damaging propellants such as butane; air displacement may be nitrogen. For liquid compositions it is essential that the effective amount of the drug and/or vaccine can be administered in an appropriate volume. For nasal administration the volume should not exceed about 1000 μl for a human subjects unless the bioadhesive, according to the invention, is used. A larger volume can be disagreeable to the patient and will drain out anteriorly through the nostrils or posteriorly toward the pharynx. The result is that a part of the drug and/or the antigen and/or the vaccine is lost from the absorption site, unless the bioadhesive, according to the invention, is used. The volume is preferably from about 20 μl to about 250 μl and preferably administered into one nostril. For the administration to the buccal and rectal area, a volume not exceeding 10 ml should be used. For the administration to the eye and the ear a volume not exceeding 300 μl should be used. For the administration to the lungs, a volume not exceeding 2 ml should be used. For the administration to the vagina, a volume not exceeding 30 ml should be used. For administration to the gastrointestinal tract a volume not exceeding 100 ml should be used.

The antigen and/or the vaccine composition may optionally comprise additional immunological adjuvants, such as lysolecithin, muramyl dipeptide, dimethylglycine, tuftsin;

immune stimulating complexes; oil emulsions; lipopolysaccharides such as MPL (3-O-deacylated monophosphoryl lipid A; mineral gels, and immunostimulating oligonucleotides. The antigens of this invention can also be incorporated into liposomes, cochleates, biodegradable polymers such as poly-lactide, poly-glycolide and poly-lactide-co-glycolides, or ISCOMS (immunostimulating complexes), and supplementary active ingredients may also be employed.

Antigens of the present invention can also be administered in combination with bacterial toxins and their attenuated derivatives as adjuvants or carrier molecules. Other suitable carrier molecules include serum albumins, keyhole limpet hemocyanin, immunoglobulin molecules, immunoglobulin, ovalbumin, polysaccharides (e.g., sepharose, agarose, cellulose), inactive virus particles, and amino acid copolymers. The antigens of the invention can also be administered in combination with cytokines, chemokines, lymphokines including, but not limited to, IL-1, II-2, II-4, II-6, II-10, II-12, II-14, II-16, IFN-γ, TGF-β, GM-CSF etc. The antigens of the invention can also be expressed in vivo after administration of DNA encoding these antigens. The antigens may include but not limited to autoimmune antigens such as for lupus, psoriasis, arthritis, MS, diabetes etc; allergic antigens such as birch, house-dust, cat, horse, bees, seafood etc; cancer-allergens and/or vaccines such as for bacterial, viral, fungal, parasite and/or prion infections.

The formulation according to the invention is especially suitable for humans, including toddlers, adolescents, teenagers, adults and elderly.

The bioadhesive effect of the bioadhesive agents of the invention has a number of important implications. The bioadhesive agent can increase the duration of mucosally administered antigens in the vaccinated organism, providing appropriate time for the antigen presenting cells to recognize the antigen. As a result, effective vaccination can be achieved with a smaller quantity of antigen than would be normally required. This reduction in the required amount of antigen may lead to more widespread use of vaccines, which are difficult and costly to prepare. Additionally, the use of adjuvants of the invention can enhance the ability of antigens which are weakly antigenic or poorly immunogenic, particularly when administered mucosally, to elicit an immune response. It may also provide for safer vaccination when the antigen is toxic at the concentration normally required for effective mucosal immunization. By reducing the amount of antigen, the risk of toxic reaction is reduced. It may also provide for safer vaccination by enabling the use of an antigen or vaccine formulation that is safe by mucosal mute but toxic by parenteral route. The vaccines that may be used together with this invention are also useful therapeutically, to reduce the number and severity of symptomatic episodes in subjects already infected with the antigen, such as autoimmune disease antigens, allergens, cancer antigens etc. The vaccine compositions may also be used as an oral booster immunization for parenterally administered antigens.

The composition according to the invention is also suitable for administration to animals such as horses, sheep, dogs, cats, cows, pigs, goats, rabbits, wild animals and laboratory animals such as mice, rats, guinea pigs, hamsters, rabbits, dogs, cats or monkeys; to birds such as chickens, turkeys, ducks, ostrich, tropical birds or wild birds; or to fish such as farm fish, e.g., salmon or aquarium fish. For animals, the concentration of each component may need to be adjusted. For fish, the formulation may be administered to the skin, the oral cavity, eyes or the gills.

The bioadhesive agent according to the invention can also be used to prolong the duration of bioactive substances in plants providing optimal time for the uptake of bioactive substances into plants. Active agents, which can be used in conjunction with the compositions of the invention, can be, e.g., herbicides, insecticides, fungicides, plant growth regulators, fertilizers and vaccines. Thus, the invention also relates to a method for the preparation of a bioadhesive composition for use in plants comprising mixing a bioadhesive agent according to the invention with an active substance selected from the group consisting of herbicides, insecticides, fungicides, plant growth regulators, fertilizers, antigens and vaccines.

Many appropriate bioactive agents are commercially available and can generally be applied at rates recommended by the supplier. The particular amount of bioactive ingredient for use in formulations of the invention will vary with the specific plant or plant growth medium with which it is to be contacted, the general location of an application, i.e., sheltered areas such as greenhouses as compared with exposed areas such as fields, and the type of formulation (e.g., aerosol, liquid, solid). The formulation can be used on any type of plant, including, but not limited to, grasses (e.g., blac kgrass, cheat grass, crabgrass, barnyard grass, goose grass, ryegrass, italian grass), crop plants (e.g., wheat, oats, sorghum, corn, sugar beets, canola, soybean, cotton, tobacco, potato, fruit trees, cucumber, tomato, banana, beans, peppers, melons) and ornamental plants (shrubs, trees, flowers). The formulation can be applied to the leaves, seeds, fruits, bark or wood, or can be admixed with the growth medium (e.g., peat or soil). The formulation can be applied to plants individually (e.g., with a spray applicator) or en masse (e.g., from an aircraft). The delivery of antigens to a plant using the adjuvant compositions of the invention can be used to immunize plants against pathogens (e.g., by the generation of plantibodies or by the activation of local and systemic acquired resistance (see, for example, Agrios, Plant Pathology, fourth edition (Academic Press, 1997), pages 108-114, and references cited therein).

The following Examples are offered for the purpose of illustrating the present invention and are not to be construed to limit the scope of this invention. The contents of all references, patents and published patent applications cited throughout this application are hereby incorporated by reference.

### EXAMPLES

### Example 1

### Demonstration of a Bioadhesive Effect of a Composition According to the Invention

Mice (BALB/c) are intranasally administered 5 μL formulation containing 1.5 μL fluorescence labelled albumin in the following formulations: (I) isotonic saline; (II) 5% PEG6-CCG (Softigen; Condea Chemie A G, Witten, Germany) which is a monoglyceride/diglyceride mixture of caprylic and capric acid containing 6 polyoxyethylene (PEG6) units in isotonic saline. At following time intervals the animals are sacrificed and the amount of radiolabelled vaccine on the nasal mucosal surface is evaluated by measuring the radioactivity on the mucosa:

Concentration
on the mucosa

| Group | 5 min | 2 h | 4 h |
|---|---|---|---|
| Saline | 80% | 3% | 0% |
| PEG6-CCG | 95% | 90% | 85% |

The results show that Softigen prolongs the duration of the concentration of albumin on the mucosal surface inside the nasal cavity.

### Example 2

### Demonstration of Prolonged Duration at the Administration Site

Mice (BALB/c) are given tetracycline in 5% PEG6-CCG, which is a monoglyceride/diglyceride of caprylic and capric acid containing 6 polyoxyethylene (PEG6) units, in isotonic saline. The formulation is administered (I) 5 μL Intranasally; (II) 5 μL orally into the mucosa of the mouth; (III) 5 μL vaginally; (IV) 5 μL rectally; and (V) 5 μL dermally. The formulation according to the invention showed a significant prolonged duration at the site of absorption.

### Example 3

### Demonstration of a Bioadhesive Effect of a Composition According to the Invention

A formulation containing 0.5%, 5% and 10% PEGS-CCG, which is a monoglyceride/diglyceride of capryllc and capric acid containing 6 polyoxyethylene (PEG6) units, in isotonic saline and a selected vaccine composition is administered as follows: (I) 1.5 IU insulin to mice; (II) 6 IU Insulin to rabbits; (III) 20 IU insulin sheep; (IV) 12 IU insulin to human volunteers; (V) 3 mg tetracycline to salmon; (VI) 3 mg tetracycline vaccine to chicken; and (VII) 3 doxycycline to potato plants. The formulations showed a prolonged duration of the substances at the site of administration in all species providing improved time for prolonged delivery of the bioactive substance.

### Example 4

### Demonstration of a Significant Improved Effect when Softigen ® is Applied as a Bioadhesive Agent According to the Invention

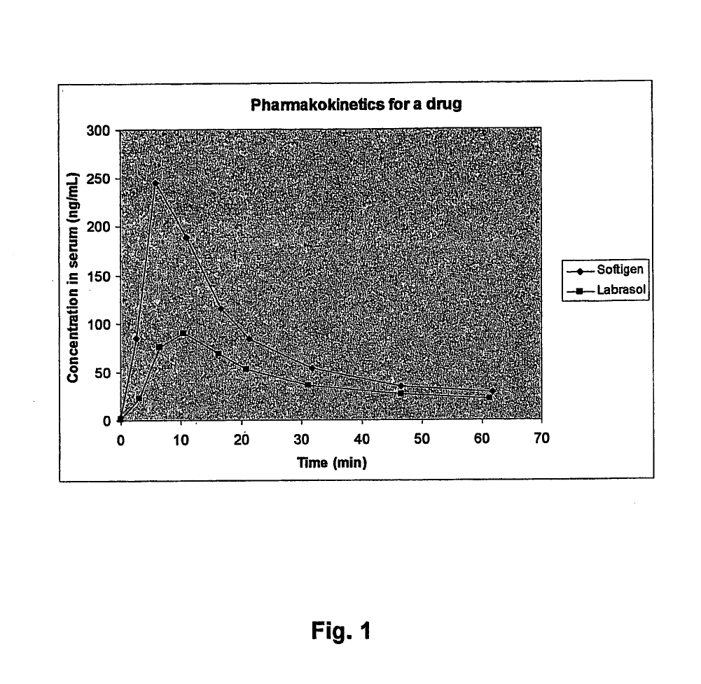

In a cross-over study, 4 rabbits received drug A in a formulation containing 1% Softigen® (Sasol GmbH, Germany) (n=4) and 1% Labrasol® (Gattefosse, France) (n=4) and were sampled for blood samples after 0, 2, 5, 10, 15, 30, 45 and 60 min. Comparison of these two formulations showed that Softigen® had AUC=4.875 and tmax=6 min whereas Labrasol® had AUC=2.552 and tmax was 10 min. See FIG. 1.

By the manufacturer Labrasol® is denoted caprylocaproyl macrogolglycerides that are mixtures, mainly of the following compounds 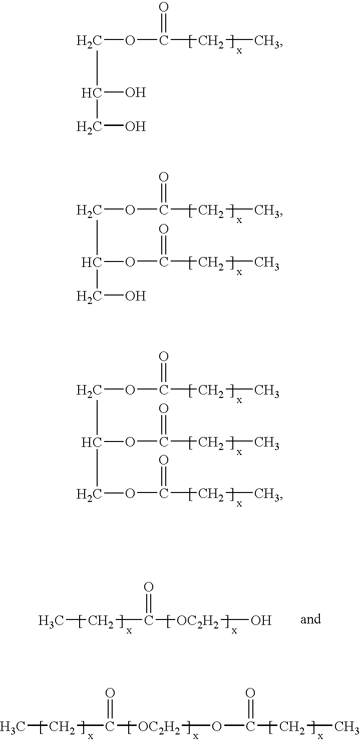
x=6 or 8 (capric and caprylic acids), y=8 on average, free glycerol is less 5%. Accordingly, Labrasol® is not a composition according to the present invention as it is not included in the definition of compounds of formula I

In contrast to Labrasol®, Softigen is a composition according to formula I.

Softigen® is denoted macrogol-6-glycerol-caprylocaprate and is mixtures, mainly of the following compounds: 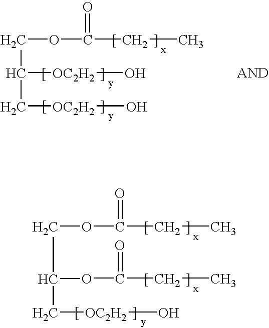
x=6 or 8 (capric and caprylic acids) and the sum of y on each molecule is on average 6.

Softigen consists of octanoic/decanoic macrogol-6 glycerides, which are made by ethoxylation of mono and diglycerides of octanoic and decanoic acids. Labrasol on the other hand is a mixture of macrogol-8 octanoic/decanoic mono or diester and mono-, di- and triesters of glycerol. This composition results from the partial alcoholysis of the corresponding triglycerides using macrogol 400.

In the following table is given a comparison of Softigen® and Labrasol®.

| Trade name | Softigen 767 ® | Labrasol ® |
|---|---|---|
| Source | Sasol GmbH | GATTEFOSSE |
| | www.condea.com | http://www.gattefosse.com |
| | Softigen 767 ® | Labrasol ® |
| Chemical | Macrogol-6-glycerol | Caprylocaproyl macrogol- |
| composition | caprylocaprate is a mixture | 8 glycerides as a well |
| | of mono and diesters, made | defined mixture of mono-, |
| | of polyoxyethyl glycerol | di- and triglycerides and |
| | ethers | mono and di-fatty acid |
| | | esters of polyethylene |
| | | glycol |
| Ph. Eur. | Macrogol-6-glycerol | Caprylocaproyl |
| | caprylocaprate | macrogolglycerides |
| | 01/2000:1443 | 01/2002:1184 |
| Average # of | Average number of ethoxy | Eight (8). Applies only for |
| ethoxy units | units per molecule is | the mono and di-fatty acid |
| | six (6). | esters of polyethylene |
| | | glycol (PEG 400) |
| Methods of | Ethoxylation of mono and | The substance is made |
| synthesis | di glycerides from capric- | by an alcoholysis/esterifi- |
| | and caprylic acid that are | cation reaction using |
| | synthesized through | medium chain |
| | esterificatlon of glycerol | triglycerides from coconut |
| | with destilled coconut or | oil and PEG 400 as |
| | palm-core fatty acids | starting material. |
| Viscosity @ | Ca. 160 mPa s | 80-110 mPa s |
| 20° C. | | |
| HPB value | Ca. 19 | 14 |

1-36. Canceled.

37. A method of enhancing the bioadhesion of a liquid pharmaceutical composition suitable for administration to a mucus membrane, comprising preparing a pharmaceutical composition suitable for administration to a mucus membrane comprising an active agent and one or more mono- or diglycerides having Formula (I): 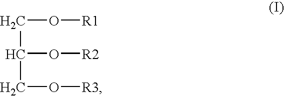
wherein R1, R2 and R3 are selected from the group consisting of C6 to C26 fatty acid, polyethylene glycol (PEG) polymers and hydrogen, provided that at least one of R1, R2 and R3 is a C6-C26 fatty acid residue and at least one of R1, R2 and R3 is a PEG polymer residue, provided that the pharmaceutical composition comprises an active substance that is not an antigen.

38. The method of claim 37, wherein the PEG polymer contains from 2 to about 30 residues of ethylene glycol or derivatives thereof.

39. The method of claim 38, wherein the PEG polymer has from 3 to about 6 residues.

40. The method of claim 37, wherein the pharmaceutical composition comprises a mixture of mono- and diglycerides.

41. The method of claim 40, wherein the volume to volume ratio of monoglycerides to diglycerides is from about 0.1 to 99.9 to about 99.9 to 0.1.

42. The method of claim 37, wherein at least one of R1, R2 and R3 is selected from saturated C6-C26 fatty acids.

43. The method of claim 42, wherein at least one of R1, R2 and R3 is a C6, C8 or C10 fatty acid.

44. The method of claim 37, wherein the total concentration of glycerides in the pharmaceutical composition is at least about 90% by weight.

45. The method of claim 44, wherein the pharmaceutical composition comprises from about 5 to about 95% by weight of the diglycerides represented by Formulas (II) and (III): 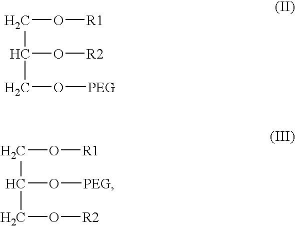
and from about 5% to about 95% by weight of the monoglycerides represented by Formulas (IV) and (V):

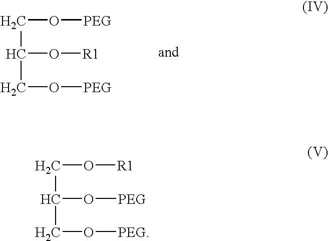

46. The method of claim 37, wherein the mono-or diglyceride is a monoglyceride having Formula (VI): 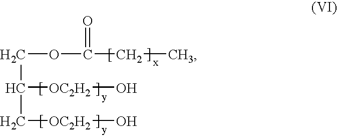
wherein x is an integer from about 4 to about 20 and y is an integer from 2 to about 30.

47. The method of claim 46, wherein x is 6 or 8 and y is independently 3 or 6.

48. The method of claim 37, wherein the mono- or diglyceride is a diglyceride having Formula (VII): 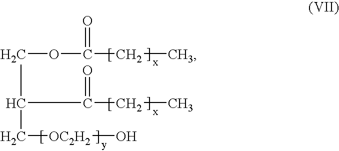
wherein x is an integer from about 4 to about 20 and y is an integer from 2 to about 30.

49. The method of claim 48, wherein x is independently 6 or 8 and y is 3 or 6.

50. The method of claim 37, wherein the active substance is a drug, protein, peptide, ion, gene, plasmid, antisense molecule, oligonucleotide, diagnostic, antibody, allergen, environmental toxin, or narcotic.

51. A method of preparing a bioadhesive liquid pharmaceutical composition suitable for administration to a mucus membrane, comprising mixing an active substance and optionally one or more pharmaceutically acceptable excipients with a bioadhesive agent having Formula (I): 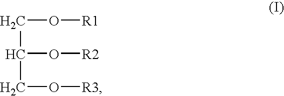
wherein R1, R2 and R3 are selected from the group consisting of C6 to C26 fatty acid, polyethylene glycol (PEG) polymers and hydrogen, provided that at least one of R1, R2 and R3 is a C6-C26 fatty acid residue and at least one of R1, R2 and R3 is a PEG polymer residue, provided that the active substance is not an antigen.

52. The method of claim 51, wherein the active substance is a drug, protein, peptide, ion, gene, plasmid, antisense molecule, oligonucleotide, diagnostic, antibody, allergen, environmental toxin, or narcotic.

53. The method of claim 51, wherein the active substance is in a particulate form.

54. The method of claim 51, wherein the active substance is in a dissolved form.

55. The method of claim 51, wherein the bioadhesive agent comprises about 0.005% to about 99% by weight of the pharmaceutical composition.

56. The method of claim 51, wherein the pharmaceutical composition comprises one or more pharmaceutically acceptable excipients selected from the group consisting of surfactants, absorption promoters, water absorbing polymers, substances which inhibit enzymatic degradation, alcohols, organic solvents, oils, pH-controlling agents, solubilizers, stabilizers, HLB-controlling agents, viscosity controlling agents, preservatives, osmotic pressure controlling agents, propellants, air displacement, water, and mixtures thereof.

57. A method of preparing a bioadhesive composition suitable for use in plants, comprising mixing an active substance selected from the group consisting of herbicides, insecticides, fungicides, plant growth regulators, fertilizers, antigens and vaccines with one or more mono- or diglycerides having Formula (I): 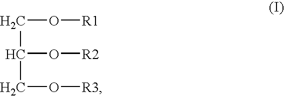
wherein R1, R2 and R3 are selected from the group consisting of C6 to C26 fatty acid, polyethylene glycol (PEG) polymers and hydrogen, provided that at least one of R1, R2 and R3 is a C6-C26 fatty acid residue and at least one of R1, R2 and R3 is a PEG polymer residue.

58. A method of treating a plant with an active substance, comprising treating the plant with a composition comprising an active substance selected from the group consisting of herbicides, insecticides, fungicides, plant growth regulators, fertilizers, antigens and vaccines and one or more mono- or diglycerides having Formula (I): 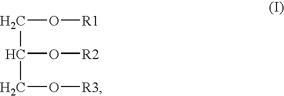
wherein R1, R2 and R3 are selected from the group consisting of C6 to C26 fatty acid, polyethylene glycol (PEG) polymers and hydrogen, provided that at least one of R1, R2 and R3 is a C6-C26 fatty acid residue and at least one of R1, R2 and R3 is a PEG polymer residue.

59. A method of prolonging the duration for which an active substance resides on a mucus membrane, comprising administering a pharmaceutical composition suitable for administration to a mucus membrane comprising an active substance and one or more mono- or diglycerides having Formula (I): 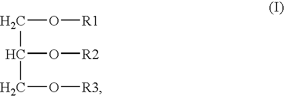
wherein R1, R2 and R3 are selected from the group consisting of C6 to C26 fatty acid, polyethylene glycol (PEG) polymers and hydrogen, provided that at least one of R1, R2 and R3 is a C6-C26 fatty acid residue and at least one of R1, R2 and R3 is a PEG polymer residue, provided that the pharmaceutical composition comprises an active substance that is not an antigen.

60. A method of administering a pharmaceutical composition to the gills of a fish, comprising administering to the gills of a fish a pharmaceutical composition comprising an active agent and one or more mono- or diglycerides having Formula (I): 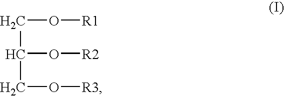
wherein R1, R2 and R3 are selected from the group consisting of C6 to C26 fatty acid, polyethylene glycol (PEG) polymers and hydrogen, provided that at least one of R1, R2 and R3 is a C6-C26 fatty acid residue and at least one of R1, R2 and R3 is a PEG polymer residue.

61. A method of prolonging the residence time of an active agent on a surface, comprising administering a first pharmaceutical composition comprising an active agent and administering a second pharmaceutical composition comprising one or more mono- or diglycerides having Formula (I): 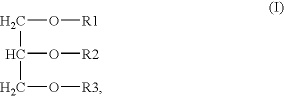
wherein R1, R2 and R3 are selected from the group consisting of C6 to C26 fatty acid, polyethylene glycol (PEG) polymers and hydrogen, provided that at least one of R1, R2 and R3 is a C6-C26 fatty acid residue and at least one of R1, R2 and R3 is a PEG polymer residue.

62. The method of claim 61, wherein the first pharmaceutical composition and the second pharmaceutical composition are administered at the same time.

63. The method of claim 61, wherein the first pharmaceutical composition and the second pharmaceutical composition are administered at different times.
